(19) United States
(12) Patent Application Publication
(10) Pub. No.: US 2020/0066412 A1
(43) Pub. Date: Feb. 27, 2020

(54) VALIDATING EFFICACY OF MEDICAL ADVICE

(71) Applicant: INTERNATIONAL BUSINESS MACHINES CORPORATION, Armonk, NY (US)

(72) Inventors: Michael Bender, Rye Brook, NY (US); Gregory J. Boss, Saginaw, MI (US); Jeremy R. Fox, Georgetown, TX (US)

(21) Appl. No.: 16/106,707

(22) Filed: Aug. 21, 2018

(51) Int. Cl. G16H 70/40; G16H 10/60; G16H 20/10

(52) U.S. Cl. CPC G16H 70/40; G16H 70/60; G16H 20/10; G16H 10/60

(57) ABSTRACT

A computer-implemented method includes: receiving, by a computer device, registration of a user; receiving, by the computer device, a treatment plan prescribed for the user; tracking, by the computer device, how the treatment plan is being followed by the user; tracking, by the computer device, a change in health of the user; and providing, by the computer device, a report based on the tracking how the treatment plan is being followed and the tracking the change in health.

### BACKGROUND

The present invention relates generally to validating efficacy of medical advice and, more particularly, to crowd-sourcing the validation of efficacy of medical advice.

Medical professionals (e.g., doctors, nurses, etc.) provide medical advice to patients as a matter of course. The advice can include instructions, recommendations, prescriptions, etc. Sometimes a patient has a follow-up visit with a medical professional during which the medical professional can obtain feedback directly for the patient about how well the advice addressed the issue for which the advice was given. Other times, patients do not have follow-up visits with a medical professional that has previously provided such advice.

### SUMMARY

In a first aspect of the invention, there is a computer-implemented method including: receiving, by a computer device, registration of a user; receiving, by the computer device, a treatment plan prescribed for the user; tracking, by the computer device, how the treatment plan is being followed by the user; tracking, by the computer device, a change in health of the user; and providing, by the computer device, a report based on the tracking how the treatment plan is being followed and the tracking the change in health.

In another aspect of the invention, there is a computer program product including a computer readable storage medium having program instructions embodied therewith. The program instructions are executable by a computing device to cause the computing device to receive registration of a user; receive a treatment plan prescribed for the user; track how the treatment plan is being followed by the user; track a change in health of the user; and provide a report based on the tracking how the treatment plan is being followed and the tracking the change in health.

In another aspect of the invention, there is system including a processor, a computer readable memory, and a computer readable storage medium. The system includes program instructions to receive registration of a user; program instructions to receive a treatment plan prescribed for the user; program instructions to track how the treatment plan is being followed by the user; program instructions to track a change in health of the user; and program instructions to provide a report based on the tracking how the treatment plan is being followed and the tracking the change in health. The program instructions are stored on the computer readable storage medium for execution by the processor via the computer readable memory.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 depicts a computer infrastructure according to an embodiment of the present invention.

FIG. 2 shows a block diagram of an exemplary environment in accordance with aspects of the invention.

FIG. 3 shows a flowchart of an exemplary method in accordance with aspects of the invention.

FIG. 4 shows a flowchart of an exemplary method in accordance with aspects of the invention.

### DETAILED DESCRIPTION

The present invention relates generally to validating efficacy of medical advice and, more particularly, to crowd-sourcing the validation of efficacy of medical advice. According to aspects of the invention, a system obtains feedback about medical advice that is provided to users. In embodiments, the feedback includes data defining how closely the advice was followed and how well the advice, when followed, addressed the problem for which the advice was provided. In embodiments, the feedback is obtained automatically (e.g., via Internet of Thing (IoT) devices) and via user input (e.g., via questionnaire). In accordance with aspects of the invention, such feedback is obtained for a large population of users, and machine learning techniques are used to determine compliances rates and effectiveness rates for different categories of medical advice. In this manner, implementations of the invention use crowd sourcing and machine learning to identify categories of medical advice that are likely to be followed by users, as well as categories of medical advice that, when followed, are likely to produce positive results for users.

It is all too common for medical professionals to provide medical advice to a user and then never hear back from the user about how well the medical advice worked in addressing the user's situation. For example, during an office visit, a doctor may prescribe a prescription antihistamine to a user suffering from seasonal allergies. In this example, unless the user has a follow-up appointment with the doctor at a later date, the doctor has no way of knowing if the user actually took the antihistamine in the manner prescribed, or if the antihistamine (if taken by the user in the manner prescribed) worked to provide a positive result for the user's allergies. This is a feedback void that doctors are experiencing, as they don't know the outcome of a recommendation, or even if the recommendation was followed. This feedback void prevents doctors from learning how effective a particular recommendation is for a category of the user population.

Implementations of the invention address this problem by learning and rating the efficacy of a doctor's recommendation by capturing feedback from patients. In embodiments, the feedback comes from manual entry and/or IoT devices and includes tracking that the instructions were followed and the results of the treatment. In embodiments, after the data is made available across a large spectrum of individuals, it is categorized and analyzed to determine compliance rates and efficacy rates for different types of medical advice. In one embodiment, the compliance rates and efficacy rates are provided to medical professionals as learning tools, i.e., so that medical professionals learn the advice provides the best outcome for a particular problem. In another embodiment, the compliance rates and efficacy rates are provided to the public to increase public awareness about which advice provides the best outcome for a particular problem.

Aspects of the invention are illustrated using the following four exemplary use cases. These four use cases are for illustrative purposes, and embodiments of the invention are not limited to these four use cases. In a first use case, a doctor prescribes antihistamines for an individual suffering from allergies. A validation system in accordance with aspects of the invention tracks that the prescription was filled, and in this case, filled with a smart pill bottle. In this use case, the individual enters feedback on a daily basis on how they feel after starting the prescription treatment. After three days, significant improvement is seen in the individual's condition (i.e., allergies). The validation system in accordance with aspects of the invention uses the data (e.g., user condition, medical advice, how closely followed, results) in machine learning based determination of compliance rates and efficacy rates of different types of medical advice.

In a second use case, a doctor prescribes antihistamines for an individual suffering from allergies. A validation system in accordance with aspects of the invention tracks that the prescription was filled, and in this case, filled with a smart pill bottle. In this use case, the individual enters feedback on a daily basis on how they feel after starting the prescription treatment. After three days, no improvement is seen in the individual's condition (i.e., allergies). Comparing the instructions to those executed, the system determines that this individual took only the first one of the prescribed doses of antihistamine, and then stopped taking the antihistamine after the first does. The validation system in accordance with aspects of the invention uses the data (e.g., user condition, medical advice, how closely followed) in machine learning based determination of compliance rates, but excludes this data from determining efficacy rates since the individual did not follow the prescribed treatment.

In a third use case, a doctor prescribes antihistamines for an individual suffering from allergies. A validation system in accordance with aspects of the invention tracks that the prescription was not filled. The patient continues to complain on social media that their allergies are bad. The validation system in accordance with aspects of the invention uses the data (e.g., user condition, medical advice, how closely followed) in machine learning based determination of compliance rates, but excludes this data from determining efficacy rates since the individual did not follow the prescribed treatment.

In a fourth use case, a doctor prescribes antihistamines for an individual suffering from allergies. A validation system in accordance with aspects of the invention tracks that the prescription was filled, and in this case, filled with a smart pill bottle. The system determines from IoT device data that, the medical advice is not providing a positive result for the individual even though the individual is following the medical advice. For example, the system determines that the individual is taking the antihistamine as prescribed, and yet the individual's amount of sneezing is increasing while allergens in the area are decreasing. The validation system in accordance with aspects of the invention uses the data (e.g., user condition, medical advice, how closely followed, results) in machine learning based determination of compliance rates and efficacy rates of different types of medical advice. In this use case, the system also provides a notification to the doctor that the medical advice is not providing a positive result for the individual even though the individual is following the medical advice. In this manner, the doctor may institute another treatment pan for this individual.

In this manner, implementations of the invention use crowd souring to identify the efficacy of a treatment plan (e.g., medical advice). In this manner, implementations of the invention also eliminate data of a respective treatment plan from the efficacy analysis when the treatment plan was not followed. In this manner, implementations of the invention also identify treatment plans that people are likely not to follow.

Embodiments of the invention are implemented using particular machines including Internet of Things (IoT) devices. A particular embodiment is implemented using smart pill bottles, which are particular machines. Furthermore, some aspects of the invention are rooted in computer technology and cannot be performed manually, e.g., in the human mind or with pen and paper. For example, machine learning based analysis of potentially millions of data points to determine compliances rates and effectiveness rates for different categories of medical advice is necessarily rooted in computer technology and cannot be performed manually.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

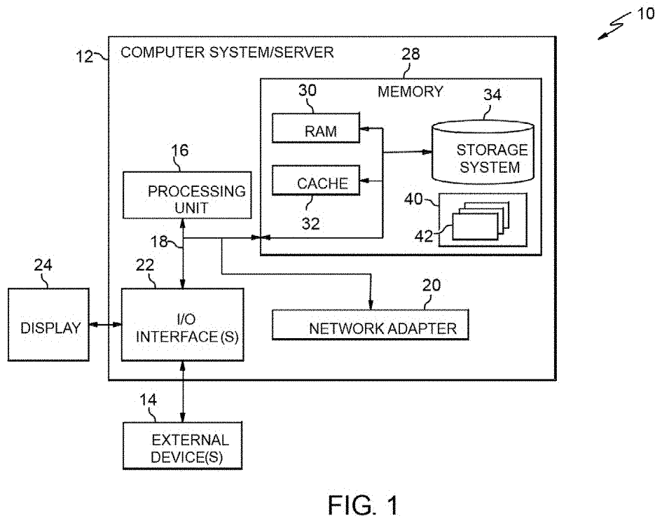

Referring now to FIG. 1, a schematic of an example of a computer infrastructure is shown. Computer infrastructure 10 is only one example of a suitable computer infrastructure and is not intended to suggest any limitation as to the scope of use or functionality of embodiments of the invention described herein. Regardless, computer infrastructure 10 is capable of being implemented and/or performing any of the functionality set forth hereinabove.

In computer infrastructure 10 there is a computer system 12, which is operational with numerous other general purpose or special purpose computing system environments or configurations. Examples of well-known computing systems, environments, and/or configurations that may be suitable for use with computer system 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 12 may be described in the general context of computer system executable instructions, such as program modules, being executed by a computer system. Generally, program modules may include routines, programs, objects, components, logic, data structures, and so on that perform particular tasks or implement particular abstract data types. Computer system 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system 12 in computer infrastructure 10 is shown in the form of a general-purpose computing device. The components of computer system 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 12 typically includes a variety of computer system readable media. Such media may be any available media that is accessible by computer system 12, and it includes both volatile and non-volatile media, removable and non-removable media.

System memory 28 can include computer system readable media in the form of volatile memory, such as random access memory (RAM) 30 and/or cache memory 32. Computer system 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system 12 may also communicate with one or more external devices 14 such as a keyboard, a pointing device, a display 24, etc.; one or more devices that enable a user to interact with computer system 12; and/or any devices (e.g., network card, modem, etc.) that enable computer system 12 to communicate with one or more other computing devices. Such communication can occur via Input/Output (I/O) interfaces 22. Still yet, computer system 12 can communicate with one or more networks such as a local area network (LAN), a general wide area network (WAN), and/or a public network (e.g., the Internet) via network adapter 20. As depicted, network adapter 20 communicates with the other components of computer system 12 via bus 18. It should be understood that although not shown, other hardware and/or software components could be used in conjunction with computer system 12. Examples, include, but are not limited to: microcode, device drivers, redundant processing units, external disk drive arrays, RAID systems, tape drives, and data archival storage systems, etc.

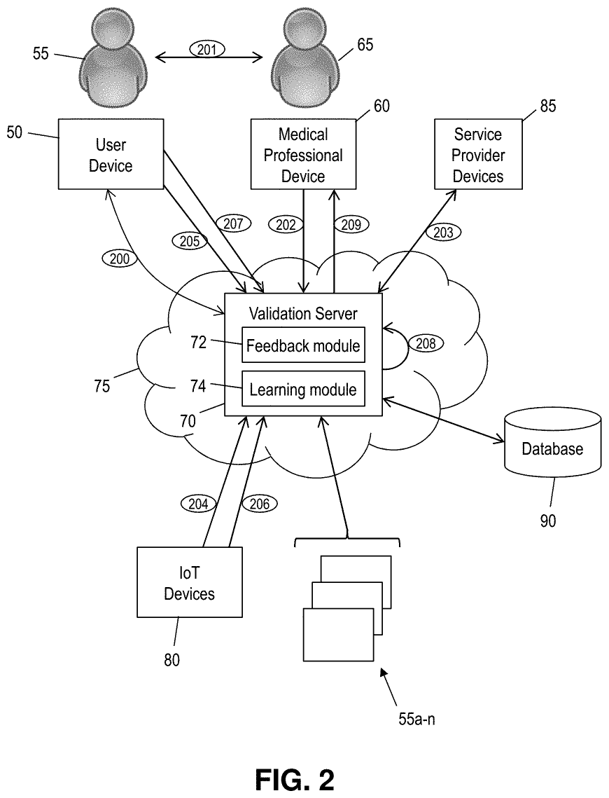

FIG. 2 shows a block diagram of an exemplary environment in accordance with aspects of the invention. In embodiments, the environment includes a user device 50 associated with a user 55 and a medical professional device 60 associated with a medical professional 65. The user device 50 and the medical professional device 60 each comprise a computer device (e.g., such as a smartphone, tablet computer, laptop computer, or desktop computer) including one or more components of computer system 12 of FIG. 1. The user device 50 and the medical professional device 60 each communicate with a validation server 70 via a network 75.

The validation server 70 comprises one or more computer servers including one or more components of computer system 12 of FIG. 1. In embodiments, the validation server 70 comprises a feedback module 72 and a machine learning module 74, each of which comprises one or more program modules 42 (of FIG. 1) configured to perform one or more processes described herein. For example, the feedback module 72, in implementations, is configured to obtain feedback data from one or more of the user device 50, IoT devices 80, and service provider devices 85. The learning module 74, in implementations, is configured to analyze the feedback data obtained by the feedback module 72 to determine compliances rates and effectiveness rates for different types of medical advice for different categories of users.

The network 75 comprises a communication network including, for example, one or more of a LAN, a WAN, and the Internet. In an embodiment, the network 75 is part of a cloud computing environment, and the processes performed by the validation server 70 are provided to the user device 50 and/or the medical professional device 60 as a cloud service (e.g., as Software as a Service (SaaS)).

Still referring to FIG. 2, the IoT devices 80 comprise IoT devices that automatically provide feedback to the feedback module 72. For example, in embodiments, the IoT devices 80 comprise a smart pill bottle which is an IoT device that is configured to publish data (to the feedback module 72) such as: the date and time that a pill is taken from the smart pill bottle, how many pills are taken from the smart pill bottle at the date and time, etc. In embodiments, the IoT devices 80 additionally or alternatively comprise wearable sensors that are configured to detect biometric data of the user 55. For example, in embodiments, the IoT devices 80 comprise one or more sensors that are configured to detect at least one of: body temperature, blood pressure, heart rate, sweat composition, sweat rate, blood oxygen level, blood glucose level, pedometer, breathing rate, coughing, sneezing, etc. The IoT devices 80 provide data to the validation server 70 via the network 75.

With continued reference to FIG. 2, in embodiments the service provider devices 85 comprise computer devices associated with third party service providers, such as a pharmacy, physical therapy provider, etc. In embodiments, the service provider devices 85 provide data to the validation server 70 via the network 75.

An exemplary implementation of the invention is illustrated by the numbered steps 200-209 of FIG. 2. At step 200, the user 55 registers with the system. In embodiments, the user 55 uses the user device 50 to register with the validation server 70. In aspects, registration includes creating a user profile, providing medical history information and demographics, and receiving a unique user identifier generated by the validation server 70. In embodiments, the unique user identifier is generated in a manner that prevents third parties from determining the actual identity (e.g., name) of the user from the unique user identifier. In this manner, the unique user identifier is a mechanism for sanitizing user data that is collected and used by the system.

Moreover, to the extent implementations of the invention collect, store, or employ personal information provided by individuals, it should be understood that such information shall be used in accordance with all applicable laws concerning protection of personal information. Additionally, the collection, storage, and use of such information may be subject to consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

At step 201, the user 55 and the medical professional 65 communicate to discuss the medical issue of the user 55. This is typically performed as a visit to the office of the medical professional 65, although the communication can also include other types of communication such as telephone, email, chat, etc. In embodiments, step 201 includes the medical professional 65 prescribing a treatment for the patient. The treatment may include any type of treatment, such as a prescription for a pharmaceutical, a suggestion to exercise for a period of time each day, an instruction to rest a particular part of the body, etc. In embodiments, step 201 includes the user 55 communicating their unique user identifier to the medical professional 65.

At step 202, the medical professional 65 uses the medical professional device 60 to provide treatment data to the validation server 70. In embodiments, the treatment data includes data such as: the unique user identifier associated with the user 55; and a description of the prescribed treatment from step 201. In the event that the treatment includes a prescription that is to be filled be another service provider (e.g., a pharmacy, physical therapist, etc.), the treatment data includes an identity of the prescribed service provider. In embodiments, the treatment data includes a code such as an ICD (International Classification of Diseases) code and/or an ICDA (International Classification of Diseases, Adapted) code.

At step 203, the validation server 70 obtains data from one or more of the service provider devices 85. In aspects, this data is used to determine whether the user is complying with the prescribed treatment. In embodiments, the validation server 70 uses the identity of the prescribed service provider included in the treatment data to poll the appropriate one or more of the service provider devices 85 to determine whether the user 55 completed the prescribed treatment. In one example, the treatment of step 201 includes a prescription for an allergy medicine, and the treatment data of step 202 includes the name of the pharmacy where the medical professional 65 called-in the prescription for the allergy medicine. In this example, the validation server 70 periodically polls the computer device of the specified pharmacy to determine whether the user 55 obtained the prescribed allergy medicine from the pharmacy. In this manner, the validation server 70 obtains data about whether the user 55 complied with an aspect of the prescribed treatment. In embodiments, the validation server 70 saves the data in a record in a database 90, wherein the record is associated with the unique user identifier associated with the user 55.

At step 204, the validation server 70 obtains data from one or more of the IoT devices 80. In embodiments, the validation server 70 uses this data from one or more of the IoT devices 80 to determine whether the user is complying with the prescribed treatment of step 201. In the example of the prescribed allergy medicine, the validation server 70 obtains data from a smart pill bottle that holds the prescribed allergy medicine, the data including when the user removes a pill from the smart pill bottle, how many pills the user removes, etc. In another example, suppose that the prescribed treatment of step 201 includes instructions to walk or jog for 30 minutes each day. In this example, the validation server 70 obtains data from a wearable device such as a pedometer. Aspects of the invention are not limited to these examples, and the validation server 70 may obtain any suitable type of data from one or more of the IoT devices 80. In this manner, the validation server 70 obtains data that can be analyzed to determine whether the user 55 complied with an aspect of the prescribed treatment. In embodiments, the validation server 70 saves the data in the record associated with the unique user identifier in the database 90.

At step 205, the validation server 70 obtains data from the user device 50. In embodiments, the validation server 70 provides a questionnaire to the user 55 via the user device 50, the questionnaire including questions about how the user is complying with the prescribed treatment of step 201. In this manner, the validation server 70 obtains data that can be analyzed to determine whether the user 55 complied with an aspect of the prescribed treatment. In embodiments, the validation server 70 saves the data in the record associated with the unique user identifier in the database 90.

At step 206, the validation server 70 obtains data from one or more of the IoT devices 80. In embodiments, the validation server 70 uses this data from one or more of the IoT devices 80 to determine an effectiveness of the prescribed treatment of step 201. In the example of the prescribed allergy medicine, the validation server 70 obtains data from a wearable sensor that detects sneezing of the user. In the example of the prescribed walking or jogging for 30 minutes per day, the validation server 70 obtains data from a wearable sensor that detects the resting heart rate of the user. Aspects of the invention are not limited to these examples, and the validation server 70 may obtain any suitable type of data from one or more of the IoT devices 80. In this manner, the validation server 70 obtains data that can be analyzed to determine a measure of effectiveness of the prescribed treatment. In embodiments, the validation server 70 saves the data in the record associated with the unique user identifier in the database 90.

At step 207, the validation server 70 obtains data from the user device 50. In embodiments, the validation server 70 provides a questionnaire to the user 55 via the user device 50, the questionnaire including questions about the effectiveness of the prescribed treatment of step 201. In this manner, the validation server 70 obtains data that can be analyzed to determine a measure of effectiveness of the prescribed treatment. In embodiments, the validation server 70 saves the data in the record associated with the unique user identifier in the database 90.

At step 208, the validation server 70 analyzes the data from steps 203-205 to determine a measure of the compliance of the user 55 with the prescribed treatment of step 201. In embodiments, the feedback module 72 accesses the data from steps 203-205 from the database 90 and determines the user's measure of compliance based on this data. Any desired scoring algorithms may be programmed in the feedback module 72 to determine the measure of compliance. In embodiments, the measure of compliance is a numerical value within a predefined range (e.g., a numerical score in the range of 0 to 100, with a score of 0 representing lowest compliance and a score of 100 representing highest compliance), although any desired measure may be used.

Also at step 208, the validation server 70 analyzes the data from steps 206-207 to determine a measure of the efficacy of the prescribed treatment of step 201 for the user 55. In embodiments, the feedback module 72 accesses the data from steps 206-207 from the database 90 and determines determine a measure of the efficacy of the prescribed treatment based on this data. Any desired scoring algorithms may be programmed in the feedback module 72 to determine the measure of the efficacy. In embodiments, the measure of efficacy is a numerical value within a predefined range (e.g., a numerical score in the range of 0 to 100, with a score of 0 representing lowest efficacy and a score of 100 representing highest efficacy), although any desired measure may be used.

At step 209, the validation server 70 provides a report to the medical professional device 60. In embodiments, the report includes the measure of compliance and the measure of the efficacy determined at step 208. In this manner, the medical professional 65 is provided with useful feedback about the treatment that was prescribed at step 201. For example, if the medical professional 65 decides that the determined measure of compliance is not satisfactory, then the medical professional 65 may prescribe a different treatment and restart the process. In another example, if the medical professional 65 decides that the determined measure of efficacy is not satisfactory, then the medical professional 65 may prescribe a different treatment and restart the process. In another example, if the medical professional 65 decides that the determined measure of compliance and the determined measure of efficacy are both satisfactory, then the medical professional 65 may make no changes to the treatment that was prescribed at step 201.

The example illustrated in FIG. 2 has been described thus far with respect to a single user, i.e., user 55. However, implementations of the invention are configured to perform the steps described herein (e.g., steps 200-209) for plural different users being treated by plural different medical professionals. The plural different users are represented by reference number 55a-n in FIG. 2. In embodiments, each time one of the users 55a-n is prescribed a treatment (e.g., at step 201), the system collects data (e.g., at steps 203-207) that is used to determine compliance and efficacy of the prescribed treatment. Each instance of a prescribed treatment and the data collected for that prescribed treatment may be stored in a respective record in the database 90.

According to aspects of the invention, the learning module 74 of the validation server 70 is configured to analyze the plural records in the database 90 (e.g., for the plural prescribed treatments) to determine crowd-sourced compliance rates and efficacy rates for different types of prescribed treatment. In embodiments, the crowd-sourced compliance rates and efficacy rates are categorized according to aspects of the available data, which includes the patient medical history (e.g., age, gender, weight, past medical conditions, etc.), ICD and/or ICDA codes, and the prescribed treatment. For example, the learning module 74 may analyze the data stored in the database 90 to determine a crowd-sourced compliance rate and efficacy rate of a prescribed allergy medicine for people aged 31-40 for ICD code J30.2 (i.e., for seasonal allergic rhinitis). In another example, the learning module 74 may analyze the data stored in the database 90 to determine a crowd-sourced compliance rate and efficacy rate of the same prescribed allergy medicine for people aged 41-50 for ICD code J30.2. In another example, the learning module 74 may analyze the data stored in the database 90 to determine a crowd-sourced compliance rate and efficacy rate of a different prescribed allergy medicine for people aged 31-40 for ICD code J30.2. These examples are not intended to limit aspects of the invention, and the learning module 74 may determine a crowd-sourced compliance rate and efficacy rate for any available category or categories of data.

With continued reference to the learning module 74 of the validation server 70, in embodiments the learning module 74 eliminates some data from the population of data when determining a crowd-sourced efficacy rate for a prescribed treatment. In embodiments, when analyzing the data from the database 90 for determining a crowd-sourced efficacy rate for a prescribed treatment, the learning module 74 analyzes data from users whose individual measure of compliance is greater than a threshold value and omits data from users whose individual measure of compliance is less than the threshold value. In embodiments, the threshold value is a value set by a system user such as a system administrator.

In embodiments, the crowd-sourced compliance rate is a numerical value indicating a percentage of users who followed the prescribed treatment. In one embodiment, the crowd-sourced compliance rate is a numerical value indicating a percent of users whose individual measure of compliance exceeds the threshold value. In embodiments, the crowd-sourced efficacy rate is a numerical value indicating a percentage of users who achieved beneficial results after following the prescribed treatment.

In accordance with further aspects of the invention, the learning module 74 of the validation server 70 is configured to use machine learning to recommend a treatment plan for the user 55. In embodiments, the validation server 70 applies machine learning techniques to the data of plural users stored in the database 90 to: (i) identify a population of users 55a-n who are in a same category as the user 55 (e.g., of users 55a-n who have the same or similar medical history and/or demographics as the user 55) and who have the same medical condition (e.g., as identified by ICD or ICDA codes) as the user 55; and (ii) identify a treatment plan prescribed amongst the identified population of users that has a highest combined measure of crowd-sourced compliance rate and crowd-sourced efficacy rate. The treatment plan identified in this manner is tailored to the user 55 (e.g., by analyzing only data from other users 55a-n who have the same or similar medical history and/or demographics as the user 55 and who have the same medical condition as the user 55) and is selected based on its optimum combined measure of crowd-sourced compliance rate and crowd-sourced efficacy rate.

In accordance with aspects of the invention, the report provided to the medical professional 65 at step 209 includes the determined crowd-sourced compliance rate and efficacy rate for the treatment that the medical professional 65 prescribed for the user 55 at step 201, in addition to the determined measures of compliance and efficacy for the individual user 55 for the prescribed treatment. In embodiments, the crowd-sourced compliance rate and efficacy rate that are provided in the report are for one or more categories that correspond to the patient medical history of the user 55, such as age, gender, past medical conditions, etc. In this manner, implementations of the invention advantageously provide the medical professional 65 with feedback as to both: (i) the compliance and effectiveness of the prescribed treatment for this particular user 55, and (ii) the compliance and effectiveness of the prescribed treatment across a population of patients that are similarly situated to the user 55. In embodiments, the report at step 209 includes the recommend treatment plan determined using machine learning.

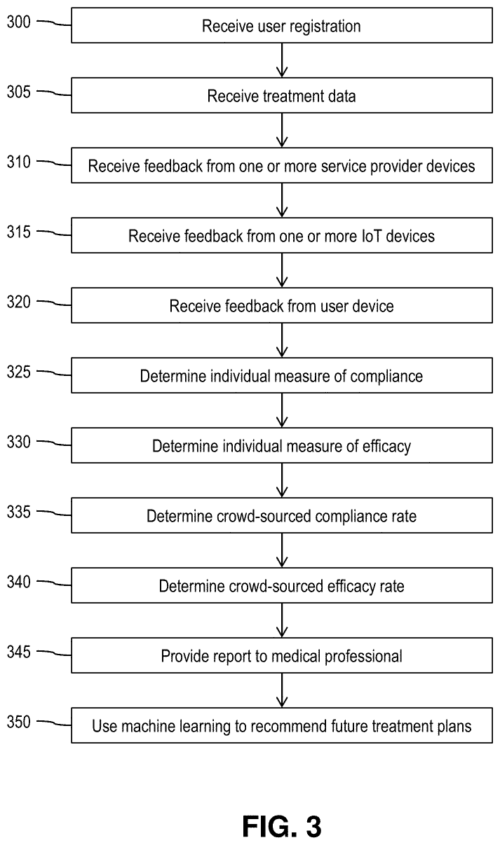

FIG. 3 shows a flowchart of an exemplary method in accordance with aspects of the present invention. In embodiments, steps of the method are carried out in the environment of FIG. 2 and are described with reference to elements depicted in FIG. 2.

At step 300, the system receives user registration. In embodiments, and as described with respect to FIG. 2, the validation server 70 receives user registration data from the user device 50, such as medical history, demographics, etc. In embodiments, step 305 includes the server 70 generating a unique user identifier for the user so that the user's stored data is sanitized and the user remains anonymous in the system.

At step 305, the system receives treatment data. In embodiments, and as described with respect to FIG. 2, the validation server 70 receives treatment data from the medical professional device 60. In embodiments, the treatment data includes: the unique user identifier associated with the user; a description of the prescribed treatment; an identity of the prescribed service provider; and a code such as an ICD (International Classification of Diseases) code and/or an ICDA (International Classification of Diseases, Adapted) code.

At step 310, the system receives feedback from one or more service provider devices. In embodiments, and as described with respect to FIG. 2, the validation server 70 receives data from one or more service provider devices 85, where the data indicates whether the user performed a prescribed aspect of the treatment (e.g., filled a prescription, attended physical therapy, etc.). In embodiments, step 310 includes the server 70 periodically polling the one or more service provider devices 85 to obtain the data.

At step 315, the system receives feedback from one or more IoT devices. In embodiments, and as described with respect to FIG. 2, the validation server 70 receives data from one or more IoT devices 80. At step 320, the system receives feedback from the user device. In embodiments, and as described with respect to FIG. 2, the validation server 70 receives data from the user device 50.

At step 325, the system determines an individual measure of compliance for this user for the prescribed treatment. In embodiments, and as described with respect to FIG. 2, the validation server 70 analyzes the data obtained from one or more of the service provider devices 85, IoT devices 80, and the user device 50 to determine a measure of compliance for this user for the prescribed treatment.

At step 330, the system determines an individual measure of efficacy for this user for the prescribed treatment. In embodiments, and as described with respect to FIG. 2, the validation server 70 analyzes the data obtained from one or more of the IoT devices 80 and the user device 50 to determine a measure of efficacy for this user for the prescribed treatment.

At step 335, the system determines crowd-sourced compliance rate for the prescribed treatment. In embodiments, and as described with respect to FIG. 2, the validation server 70 analyzes the data associated with plural users having the same prescribed treatment, and determines a compliance rate (e.g., a percent of the users that complied with the prescribed treatment).

At step 340, the system determines crowd-sourced efficacy rate for the prescribed treatment. In embodiments, and as described with respect to FIG. 2, the validation server 70 analyzes the data associated with plural users having the same prescribed treatment, and who complied with the prescribed treatment. Based on this analysis the validation server 70 determines an efficacy rate for the prescribed treatment. In embodiments, the crowd-sourced compliance rate and the crowd-sourced efficacy rate are determined for categories of users who have the same or similar medical history and/or demographics as the user who is the subject of the treatment plan at step 305.

At step 345, the system provides a report to the medical professional who prescribed the treatment at step 305. In embodiments, and as described with respect to FIG. 2, the validation server 70 sends a report card to the medical professional device 60 of the medical professional who prescribed the treatment. In embodiments, the report card includes: the individual measure of compliance for this user for the prescribed treatment; the individual measure of efficacy for this user for the prescribed treatment; the crowd-sourced compliance rate for the prescribed treatment for users having the same or similar medical history and/or demographics as the user; and the crowd-sourced efficacy rate for the prescribed treatment for users having the same or similar medical history and/or demographics as the user.

At step 350, the system uses machine learning to recommend future treatment plans. In embodiments, and as described with respect to FIG. 2, the validation server 70 applies machine learning techniques to the data of plural users to determine a recommended treatment plan for an individual user. In embodiments, step 350 includes the validation server 70 providing the determined recommended treatment plan to the medical professional device 60 so that the medical professional can review the recommended treatment plan and relay it to the user for which it is intended.

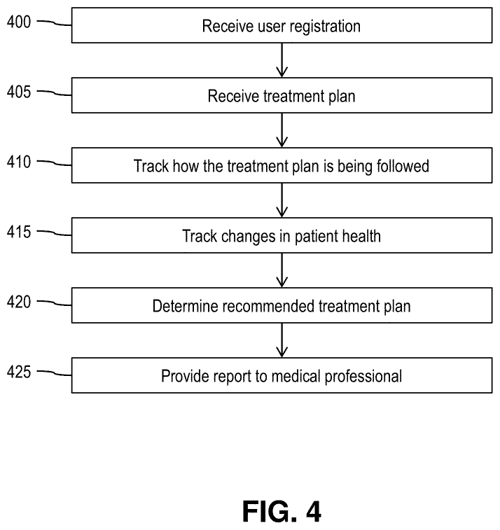

FIG. 4 shows a flowchart of an exemplary method in accordance with aspects of the present invention. In embodiments, steps of the method are carried out in the environment of FIG. 2 and are described with reference to elements depicted in FIG. 2.

At step 400, the system receives user registration. At step 405, the system receives treatment plan. In embodiments, steps 400 and 405 are performed in the manner described with respect to stapes 300 and 305, respectively.

At step 410, the system tracks how the treatment plan (i.e., the prescribed treatment in the treatment data of step 405) is being followed. In embodiments, and as described with respect to FIG. 2, the validation server 70 obtains data from various sources and determines from the data an individual measure of compliance for the user for the treatment plan.

At step 415, the system tracks changes in the patient's health. In embodiments, and as described with respect to FIG. 2, the validation server 70 obtains data from various sources and determines from the data an individual measure of efficacy for the treatment plan for the user.

At step 420, the system determines a recommended treatment plan. In embodiments, and as described with respect to FIG. 2, the validation server 70 uses machine learning to analyze data of plural users to determine a recommended treatment plan for the particular user.

At step 425, the system provides a report to the medical professional. In embodiments, and as described with respect to FIG. 2, the validation server 70 provides a report to the medical professional device 60, the report including: the individual measure of compliance for this user for the prescribed treatment; the individual measure of efficacy for this user for the prescribed treatment; the crowd-sourced compliance rate for the prescribed treatment for users having the same or similar medical history and/or demographics as the user; the crowd-sourced efficacy rate for the prescribed treatment for users having the same or similar medical history and/or demographics as the user; and the recommended treatment plan determined using machine learning at step 420.

The flowcharts shown in FIGS. 3 and 4 provide for a computer enabled method for scoring the efficacy of a treatment for a physical ailment, the method comprising: registering a patient into the system; registering the treatment plan into the system; tracking how the treatment plan is being followed; tracking any changes in the patient health; and using machine learning to recommend future treatment plans. In embodiments, the method comprises: entering the patient demographic and ICDA code for the current problem; tracking if prescriptions are filled at the pharmacy; tracking if the treatment plan is being followed; tracking changes in the patient's health; using IoT devices to capture the changes in health; using machine learning in comparing a large population to understand the efficiency of the treatment plan; comparing different subgroups based on a patient's demographic data; identifying treatment plans that are not likely to be followed; and notifying the doctor to follow up on treatment when the patient isn't improving.

Aspects of the invention described herein are useful for identifying the effectiveness of a treatment plan. In embodiments, methods of measuring the effectiveness eliminate cases by analysis of when a treatment is followed or a medication taken. The effective rate of a treatment plan that is being followed is useful information to the prescribing doctor. In addition, by virtue of having identified which treatment plans are being complied with and which are not, aspects of the invention provide feedback to the doctor as to which treatment plans are not likely to be followed by a patient. This allows the doctor to consult with the patient about their willingness to complete a treatment plan or recommend a different treatment plan.

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
receiving, by a computer device, registration of a user;
receiving, by the computer device, a treatment plan prescribed for the user;
tracking, by the computer device, how the treatment plan is being followed by the user;
tracking, by the computer device, a change in health of the user; and
providing, by the computer device, a report based on the tracking how the treatment plan is being followed and the tracking the change in health.

2. The method of claim 1, wherein the tracking how the treatment plan is being followed comprises obtaining data from at least one selected from the group consisting of:
a device of a service provider prescribed in the treatment plan;
an Internet of Things (IoT) device associated with the user; and
a user device associated with the user.

3. The method of claim 2, wherein the tracking how the treatment plan is being followed comprises determining, based on the obtained data, an individual measure of compliance of the user with respect to the treatment plan.

4. The method of claim 3, wherein the tracking the change in health comprises determining, based on the obtained data, an individual measure of efficacy of the treatment plan for the user.

5. The method of claim 1, further comprising determining a recommended treatment plan for the user.

6. The method of claim 5, wherein the determining the recommended treatment plan comprises determining crowd-sourced compliance rates and crowd-sourced efficacy rates for plural different treatment plans.

7. The method of claim 6, wherein:
the crowd-sourced compliance rate for a respective one of the plural different treatment plans is determined using a data set determined by categorization;
the crowd-sourced efficacy rate for the respective one of the plural different treatment plans is determined using a subset of data set.

8. The method of claim 7, wherein the categorization includes at least one selected from the group consisting of: patient medical history; patient demographics; and patient medical condition.

9. The method of claim 1, wherein the report includes:
a determined individual measure of compliance of the user with respect to the treatment plan;
a determined individual measure of efficacy of the treatment plan for the user;
a determined crowd-sourced compliance rate for the treatment plan; and
a determined crowd-sourced efficacy rate for the treatment plan.

10. The method of claim 9, wherein the report further includes a recommended treatment plan for the user.

11. A computer program product comprising a computer readable storage medium having program instructions embodied therewith, the program instructions executable by a computer device to cause the computer device to:
receive registration of a user;
receive a treatment plan prescribed for the user;
track how the treatment plan is being followed by the user;
track a change in health of the user; and
provide a report based on the tracking how the treatment plan is being followed and the tracking the change in health.

12. The computer program product of claim 11, wherein the tracking how the treatment plan is being followed comprises obtaining data from at least one selected from the group consisting of:
a device of a service provider prescribed in the treatment plan;
an Internet of Things (IoT) device associated with the user; and
a user device associated with the user.

13. The computer program product of claim 12, wherein the tracking how the treatment plan is being followed comprises determining, based on the obtained data, an individual measure of compliance of the user with respect to the treatment plan.

14. The computer program product of claim 13, wherein the tracking the change in health comprises determining, based on the obtained data, an individual measure of efficacy of the treatment plan for the user.

15. The computer program product of claim 11, wherein the report includes:
a determined individual measure of compliance of the user with respect to the treatment plan;
a determined individual measure of efficacy of the treatment plan for the user;
a determined crowd-sourced compliance rate for the treatment plan;
a determined crowd-sourced efficacy rate for the treatment plan; and
a recommended treatment plan for the user determined using machine learning.

16. A system, comprising:
a processor, a computer readable memory, and a computer readable storage medium;
program instructions to receive registration of a user;
program instructions to receive a treatment plan prescribed for the user;
program instructions to track how the treatment plan is being followed by the user;
program instructions to track a change in health of the user; and
program instructions to provide a report based on the tracking how the treatment plan is being followed and the tracking the change in health,
wherein the program instructions are stored on the computer readable storage medium for execution by the processor via the computer readable memory.

17. The system of claim 16, wherein the tracking how the treatment plan is being followed comprises obtaining data from at least one selected from the group consisting of:
a device of a service provider prescribed in the treatment plan;
an Internet of Things (IoT) device associated with the user; and
a user device associated with the user.

18. The system of claim 17, wherein the tracking how the treatment plan is being followed comprises determining, based on the obtained data, an individual measure of compliance of the user with respect to the treatment plan.

19. The system of claim 18, wherein the tracking the change in health comprises determining, based on the obtained data, an individual measure of efficacy of the treatment plan for the user.

20. The system of claim 16, wherein the report includes:
a determined individual measure of compliance of the user with respect to the treatment plan;
a determined individual measure of efficacy of the treatment plan for the user;
a determined crowd-sourced compliance rate for the treatment plan;
a determined crowd-sourced efficacy rate for the treatment plan; and
a recommended treatment plan for the user determined using machine learning.
